(19) United States
(12) Patent Application Publication
(10) Pub. No.: US 2020/0206299 A1
(43) Pub. Date: Jul. 2, 2020

(54) INHIBITORS OF BETA-ARRESTIN-NEUROKININ 1 RECEPTOR INTERACTIONS FOR THE TREATMENT OF PAIN

(71) Applicant: Takeda Pharmaceutical Company Limited, Osaka (JP)

(72) Inventor: Nigel Bunnett, Elwood, Victoria (AU)

(73) Assignee: Takeda Pharmaceutical Company Limited, Osaka (JP)

(21) Appl. No.: 16/078,202

(22) PCT Filed: Feb. 23, 2017
(86) PCT No.: PCT/AU2017/050158

§ 371 Date: Aug. 21, 2018

(30) Foreign Application Priority Data
Feb. 23, 2016 (AU) 2016900635

(51) Int. Cl. A61K 38/08; A61K 38/10; A61P 25/04

(52) U.S. Cl. CPC A61K 38/08; A61P 25/04; A61K 38/10

(57) ABSTRACT

The present invention relates to compounds and their uses. In particular, to compounds that inhibit the interaction between β-arrestin and the intracellular C-terminus of the activated NK1R and their use in the treatment of pain.

### FIELD OF THE INVENTION

The present invention relates to compounds and their uses. In particular, to compounds that inhibit the interaction between β-arrestin and the intracellular C-terminus of the activated NK₁R and their use in the treatment of pain.

### BACKGROUND OF THE INVENTION

G protein-coupled receptors (GPCRs) are the largest family of cell-surface receptors, participate in most pathophysiological processes, and are the target of ˜30% of therapeutic drugs (Audet, M. & Bouvier, M. Nat Chem Biol 2008, 4, 397-403). Cell-surface GPCRs interact with extracellular ligands and couple to heterotrimeric G proteins, which trigger plasma membrane delimited signals (second messenger formation, growth factor receptor transactivation, ion channel regulation). Ligand removal and receptor association with β-arrestins (βarrs) terminate plasma membrane signals.

Until recently, it was widely assumed that activation of GPCRs, subsequent down stream signaling and signal termination took place exclusively at the plasma membrane. Plasma membrane signaling is terminated within minutes of activation via phosphorylation of the receptor by GPCR kinases (GRKs) that are selective for the active ligand-bound receptor conformation. GRKs phosphorylate C-terminal S/T-rich domains of GPCRs (Sato, P. Y., et al., Physiological reviews 2015, 95, 377-404). Phosphorylated receptors then bind to βarr, which sterically prevents coupling between receptor and G-protein, thus terminating agonist-mediated G-protein activation. Parrs further promote the transfer of ligand-bound receptor from the cell surface to early endosomes via dynamin- and clathrin-dependent endocytosis. Once endocytosed, the ligand and phosphate groups are removed from the GPCR and the receptor is either rapidly redistributed to the cell membrane or it is transported to a lysosome for degradation.

Recently, however, it has been discovered that a diverse range of GPCRs do not always follow the conventional paradigm. Studies have found that following ligand binding and activation of the receptor, some cell surface GPCRs internalise and redistribute into early endosomes where heterotrimeric G protein signaling is maintained for an extended period of time. Accordingly, rather than merely acting as a conduit for GPCR trafficking to recycling or degradatory pathways, endosomes can be a vital site of signal transduction (Murphy, J. E. et al. Proc Natl Acad Sci USA 2009, 106, 17615-17622). By recruiting GPCRs and mitogen-activated protein kinases to endosomes, βarrs can mediate endosomal GPCR signaling (Murphy, J. E. et al. Proc Natl Acad Sci USA 2009, 106, 17615-17622; DeFea, K. A. et al. Proc Natl Acad Sci USA 2000, 97, 11086-11091; DeFea, K. A. et al. J Cell Biol 2000, 148, 1267-1281).

βarrs recruit diverse signaling proteins to activated receptors at plasma and endosomal membranes and are essential mediators of signaling. The MAPK cascades [ERK, c-Jun amino-terminal kinase (JNK), p38] are the most thoroughly characterized βarr-dependent signaling pathways. The first evidence that Parrs are active participants in signaling was the observation that dominant negative mutants of βarr inhibited β₂AR-induced activation of ERK1/2 (Daaka Y, et al. J Biol Chem 1998, 273, 685-688). Subsequently, Parrs were found to couple β₂AR to c-Src and mediate ERK1/2 activation (Lutterall L. M. et al. Science 1999, 283, 655-661). Parrs similarly participate in ERK1/2 signaling by other GPCRs, including neurokinin-1 receptor (NK₁R), protease-activated receptor 2 (PAR₂), angiotensin II type 1A receptor (AT_{1A}R), and vasopressin V2 receptor (V₂R). These observations led to the view that Parrs are scaffolds that couple activated GPCRs with MAPK signaling complexes. Parrs thereby mediate a second wave of GPCR signaling that is distinct from G protein-dependent signaling at the plasma membrane.

The substance P (SP) neurokinin 1 receptor (NK₁R) mediates pain and inflammation (Steinhoff, M. S. et al. Physiol Rev 2014, 94, 265-301). Although preclinical studies with antagonists of plasma membrane NK₁R signaling support its involvement in neurological and inflammatory disorders, these antagonists are ineffective treatments for chronic diseases. The NK₁R rapidly and completely redistributes to endosomes at sites of pain transmission in the spinal cord (Marvizon, J. C. et al. J Neurosci 1997, 17, 8129-8136) and inflammation in the vasculature (Bowden, J. J. et al. Proc Natl Acad Sci USA 1994, 91, 8964-8968), and is believed to internalize in patients with chronic pain and inflammation (Jarcho, J. M. et al. Pain, 2013).

It has been found that endosomal NK₁R signaling generates subcellular signals that underlie neuronal activation and hyperalgesia. Painful and pro-inflammatory stimuli, including the transient receptor potential vanilloid 1 agonist capsaicin, stimulate SP release from primary sensory nociceptors in laminae I, II of the dorsal horn, where SP stimulates NK₁R endocytosis in spinal neurons. It has been found that inhibitors of dynamin and clathrin, when injected intrathecally, inhibit NK₁R endocytosis in rats and mice and also suppress nociception.

Accordingly, inhibiting endocytosis of the activated NK₁R into early endosomes may advantageously provide a novel method for the treatment of pain.

### SUMMARY OF THE INVENTION

The present invention is predicated on the discovery that inhibiting the interaction between β-arrestin and the intracellular C-terminus of the activated NK₁R, and subsequent endocytosis of the receptor, can provide a novel method for the treatment and prevention of diseases and disorders mediated by NK₁R.

Accordingly, in one aspect the present invention provides a method for the treatment of a disease or disorder mediated by endosomal substance P (SP) or neurokinin 1 receptor (NK₁R) signaling comprising administering to a subject in need thereof a compound that inhibits interaction between β-arrestin and the intracellular C-terminus of the activated NK₁R.

In another aspect, the present invention provides use of a compound that inhibits interaction between β-arrestin and the intracellular C-terminus of the activated NK₁R in the manufacture of a medicament for the treatment of a disease or disorder mediated by endosomal substance P (SP) or neurokinin 1 receptor (NK₁R) signaling.

In a further aspect, the present invention provides a compound that inhibits interaction between β-arrestin and the intracellular C-terminus of the activated NK₁R for use in the treatment of a disease or disorder mediated by endosomal substance P (SP) or neurokinin 1 receptor (NK₁R) signaling.

In another aspect, the present invention provides a β-arrestin inhibitor of the formula:

A-D

wherein

A is a membrane permeant residue that facilitates transport of the β-arrestin inhibitor across a cellular membrane, and

D represents a fragment of one or snore phosphorylation sites on the intracellular C-terminus of NK₁R, or

pharmaceutically acceptable salts thereof.

In a further aspect, the present invention provides a β-arrestin inhibitor selected from:

A-SSSFYSNM-OH,
A-SNSKTMTE-OH,
A-LTSNGSSR-OH,
and
A-EMKS*T*RY*L-OH,

wherein

A is a membrane permeant residue that facilitates transport of the β-arrestin inhibitor across a cellular membrane; and
indicates that the amino acid is phosphorylated; or

pharmaceutically acceptable salts thereof.

In another aspect the present invention provides a method of inhibiting NK₁R endocytosis comprising contacting a cell with a β-arrestin inhibitor as herein defined.

In another aspect, the present invention provides a method for the treatment of a disease or disorder mediated by endosomal substance P (SP) or neurokinin 1 receptor (NK₁R) signaling comprising administering to a subject in need thereof an effective amount of a β-arrestin inhibitor as herein defined.

In another aspect, the present invention provides use of a β-arrestin inhibitor as herein defined in the manufacture of a medicament for the treatment of a disease or disorder mediated by endosomal SP or NK₁R signaling.

In a further aspect, the present invention provides a β-arrestin inhibitor as herein defined for use in the treatment of a disease or disorder mediated by endosomal SP or NK₁R signaling.

These and other aspects of the present invention will become more apparent to the skilled addressee upon reading the following detailed description in connection with the accompanying examples and claims.

### BRIEF DESCRIPTION OF THE DRAWINGS

FIG. 1: Graphical representations of the effects of βarr siRNA on nociception. Effects of intrathecal (i.t.) βarr siRNA. a. βarr expression. b-c. Nociception in mice. von Frey withdrawal responses of capsaicin-injected (b) or uninjected (c). *P<0.05, **P<0.01, ***P<0.001, ****P<0.0001 to control. (n) mouse number.

FIG. 2: Graphical representations of the disruption of NK₁R/βarr interaction. a. Mouse NK₁R C-terminus, indicating NK₁Rδ312 truncation and Tat-conjugated NK₁R and control peptides. b. Cell-surface ELISA: wild-type (WT) NK₁R, non-internalizing truncated variant NK₁Rδ312. b-f. SP-induced BRET: WT NK₁R-RLuc8/ or NK₁Rδ312-RLuc8/βarr1-YFP, βarr2-YFP, KRas-Venus, or Rab5a-Venus. g. SP-induced FRET. *P<0.05. Triplicate observations, n>3 experiments, n=49-99 cells. h-i. Effects of control and 3 NK₁R peptides on SP-induced NK₁R-Rluc8/βarr2-YFP BRET and NK₁R endocytosis. **P<0.01, ***P<0.001 to control.

FIG. 3: Graphical representations of the effects of NK₁R peptides on nociception. a-c. Effects of intrathecal (i.t.) NK₁R peptides on nociceptive responses to capsaicin (a), formalin (b) or complete Freund's adjuvant (CFA) (c).

### DETAILED DESCRIPTION OF THE INVENTION

By studying the substance P (SP) neurokinin 1 receptor (NK₁R) as a prototypical GPCR that robustly traffics to endosomes upon activation, it has been shown that endosomal NK₁R conveys sustained signals that underlie excitation and nociceptive transmission in spinal neurons. The concept that endosomes are platforms for compartmentalized GPCR signaling that underlies pathophysiologically important processes has implications for receptor signal-specificity and therapeutic targeting. Endosomal trafficking allows GPCRs to generate signals in subcellular compartments that may explain how different receptors that activate the same G-proteins and Parrs can specifically regulate responses. Inhibiting NK₁R endocytosis may enable targeting of signals that underlie disease-relevant processes with enhanced efficacy and selectivity.

In one aspect the present invention provides a method for the treatment of a disease or disorder mediated by endosomal substance P (SP) or neurokinin 1 receptor (NK₁R) signaling comprising administering to a subject in need thereof a compound that inhibits interaction between βarrs and the intracellular C-terminus of the activated NK₁R.

As used herein, the term “β-arrestin inhibitor” denotes a compound that inhibits the interaction between β-arrestin and the intracellular C-terminus of the activated NK₁R.

It will appreciated that the compound that inhibits the interaction between βarrs and the intracellular C-terminus of the activated NK₁R may act at any site or at multiple sites in the pathway between phosphorylation of the intracellular C-terminus of the activated NK₁R and subsequent binding of βarrs. It is envisaged that in one embodiment, inhibition of the interaction between βarrs and the intracellular C-terminus of the activated NK₁R may be achieved by administering to a subject a β-arrestin inhibitor that competes with phosphorylation sites on the intracellular C-terminus of activated NK₁R by providing an alternative site for GPCR kinase-2 (GRK2) phosphorylation, thereby reducing or ameliorating the binding of βarrs to the intracellular C-terminus of activated NK₁R and subsequent endocytosis of the receptor.

In another embodiment it is envisaged that inhibition of the interaction between βarrs and the intracellular C-terminus of the activated NK₁R may be achieved by administering to a subject a β-arrestin inhibitor that inhibits GPCR kinase-2 (GRK2) phosphorylation of the intracellular C-terminus of the active NK₁R. In one embodiment it is envisaged that the β-arrestin inhibitor that inhibits GPCR kinase-2 (GRK2) phosphorylation of the intracellular C-terminus of activated NK₁R interacts directly with GRK2, for example, by binding at the central catalytic domain of GRK2 responsible for receptor phosphorylation. In another embodiment, it is envisaged that the β-arrestin inhibitor may bind allosterically to GRK2, for example, to prevent recognition of phosphorylation sites on the intracellular C-terminus of the activated NK₁R. In yet another embodiment it is envisaged that the β-arrestin inhibitor will bind at or near phosphorylation sites within the intracellular C-terminus of NK₁R thereby preventing recognition and phosphorylation by GRK2. It is also envisaged that the compound that inhibits interaction between βarrs and the intracellular C-terminus of the activated NK₁R may act by directly interacting with β-arrestin, inhibiting it from binding to phosphorylated sites on the intracellular C-terminus of NK₁R.

In one aspect, the present invention provides use of a compound that inhibits interaction between β-arrestin and the intracellular C-terminus of the activated NK₁R in the manufacture of a medicament for the treatment of a disease or disorder mediated by endosomal substance P (SP) or neurokinin 1 receptor (NK₁R) signaling.

In a further aspect, the present invention provides a compound that inhibits interaction between β-arrestin and the intracellular C-terminus of the activated NK₁R for use in the treatment of a disease or disorder mediated by endosomal substance P (SP) or neurokinin 1 receptor (NK₁R) signaling.

In a preferred embodiment, the compound that inhibits the interaction between β-arrestin and the intracellular C-terminus of NK₁R competes with phosphorylation sites on the intracellular C-terminus of NK₁R.

In one embodiment, the β-arrestin inhibitor that competes with phosphorylation sites on the intracellular C-terminus of NK₁R is a compound of the formula:

A-D

wherein

A is a membrane permeant residue that facilitates transport of the β-arrestin inhibitor across a cellular membrane, and

D represents a fragment of one or more phosphorylation sites on the intracellular C-terminus of NK₁R, or

pharmaceutically acceptable salts thereof.

In another embodiment, the β-arrestin inhibitor that competes with phosphorylation sites on the intracellular C-terminus of NK₁R is selected from:

A-SSSFYSNM-OH,
A-SNSKTMTE-OH,
A-LTSNGSSR-OH,
and
A-EMKS*T*RY*L-OH,

wherein

A is a membrane permeant residue that facilitates transport of the β-arrestin inhibitor across a cellular membrane; and
indicates that the amino acid reside is phosphorylated; or pharmaceutically acceptable salts thereof.

In one embodiment A is a fatty acid or is the Tat membrane permeant peptide sequence YGRKKRRQRRR.

In a preferred embodiment, A is the Tat membrane permeant peptide sequence YGRKKRRQRRR. In another preferred embodiment, A is palmitic acid.

As indicated above, compounds of the invention may comprise one or more amino acid residues having side chain functionality including, but not limited to, amino acids selected from serine, tyrosine and threonine. In some embodiments, it is envisaged that the side chain functional group of the amino acid will be derivatised. In one embodiment, the side chain functional groups may be phosphorylated. In other embodiments, it is envisaged that the side chain of the amino acid will be derivatised to form a non-natural amino acid. Non-natural amino acids include any compound with both amino and carboxyl functionality. It will be understood that non-natural amino acids form part of the peptide chain through bonding via their amino and carboxyl groups.

In one aspect, the present invention provides a method for the treatment of a disease or disorder mediated by endosomal SP or NK₁R signaling comprising administering to a subject in need thereof an effective amount of a β-arrestin inhibitor as herein defined.

In another aspect, the present invention provides use of a β-arrestin inhibitor as herein defined in the manufacture of a medicament for the treatment of a disease or disorder mediated by endosomal SP or NK₁R signaling.

In a further aspect, the present invention provides a β-arrestin inhibitor as herein defined for use in the treatment of a disease or disorder mediated by endosomal SP or NK₁R signaling.

Modulation of SP-mediated NK₁R activation has been implicated in the treatment and prevention of a wide variety of disorders including depression and mood disorders, anxiety disorders, substance addiction-related disorders, alcohol-related disorders, sleep disorders, eating disorders, autism spectrum disorders, attention-deficit/hyperactivity disorder personality disorders, and cancer. Modulators of NK₁R may also be useful for the treatment and prevention of inflammation, allergic disorders, neurological disorders, emesis, pain and cancer.

In a preferred embodiment, the disease or disorder mediated by endosomal NK₁R signaling is selected from chemotherapy-induced nausea and vomiting (CINV), postoperative nausea and vomiting, affective and addictive disorders including depression and anxiety, gastrointestinal disorders including inflammatory bowl disease and irritable bowel syndrome, respiratory disorders including COPD and asthma, urogenital disorders, sensory disorders and pain including somatic pain and visceral pain, pruritus, viral and bacterial infections and proliferative disorders (cancer).

Within the context of the present invention, the term “pain” includes chronic inflammatory pain (e.g. pain associated with rheumatoid arthritis, osteoarthritis, rheumatoid spondylitis, gouty arthritis and juvenile arthritis); musculoskeletal pain, lower back and neck pain, sprains and strains, neuropathic pain, sympathetically maintained pain, myositis, pain associated with cancer and fibromyalgia, pain associated with migraine, pain associated with cluster and chronic daily headache, pain associated with influenza or other viral infections such as the common cold, rheumatic fever, pain associated with functional bowel disorders such as non-ulcer dyspepsia, non-cardiac chest pain and irritable bowel syndrome, pain associated with myocardial ischemia, post operative pain, headache, toothache, dysmenorrhea, neuralgia, fibromyalgia syndrome, complex regional pain syndrome (CRPS types I and II), neuropathic pain syndromes (including diabetic neuropathy, chemoterapeutically induced neuropathic pain, sciatica, non-specific lower back pain, multiple sclerosis pain, HIV-related neuropathy, post-herpetic neuralgia, trigeminal neuralgia) and pain resulting from physical trauma, amputation, cancer, toxins or chronic inflammatory conditions. In a preferred embodiment the pain is somatic pain or visceral pain.

Known solid or solution phase techniques may be used in the synthesis of the compounds of the present invention, such as coupling of the N- or C-terminus to a solid support (typically a resin) followed by step-wise synthesis of the linear peptide. Protecting group chemistries for the protection of amino acid residues, including side chains, are well known in the art and may be found, for example, in: Theodora W. Greene and Peter G. M. Wuts, Protecting Groups in Organic Synthesis (Third Edition, John Wiley & Sons, Inc, 1999), the entire contents of which is incorporated herein by reference.

It will be understood that the compounds of the present invention may exist in one or more stereoisomeric forms (e.g. diastereomers). The present invention includes within its scope all of these stereoisomeric forms either isolated (in, for example, enantiomeric isolation), or in combination (including racemic mixtures and diastereomic mixtures). The present invention contemplates the use of amino acids in both L and D forms, including the use of amino acids independently selected from L and D forms, for example, where the peptide comprises two serine residues, each serine residue may have the same, or opposite, absolute stereochemistry. Unless stated otherwise, the amino acid is taken to be in the L-configuration.

The invention thus also relates to compounds in substantially pure stereoisomeric form with respect to the asymmetric centres of the amino acid residues, e.g., greater than about 90% de, such as about 95% to 97% de, or greater than 99% de, as well as mixtures, including racemic mixtures, thereof. Such diastereomers may be prepared by asymmetric synthesis, for example, using chiral intermediates, or mixtures may be resolved by conventional methods, e.g., chromatography, or use of a resolving agent.

Where the compounds of the present invention require purification, chromatographic techniques such as high-performance liquid chromatography (HPLC) and reverse-phase HPLC may be used. The peptides may be characterised by mass spectrometry and/or other appropriate methods.

Where the compound comprises one or more functional groups that may be protonated or deprotonated (for example at physiological pH) the compound may be prepared and/or isolated as a pharmaceutically acceptable salt. It will be appreciated that the compound may be zwitterionic at a given pH. As used herein the expression “pharmaceutically acceptable salt” refers to the salt of a given compound, wherein the salt is suitable for administration as a pharmaceutical. Such salts may be formed, for example, by the reaction of an acid or a base with an amine or a carboxylic acid group respectively.

Pharmaceutically acceptable acid addition salts may be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

Pharmaceutically acceptable base addition salts may be prepared from inorganic and organic bases. Corresponding counter ions derived from inorganic bases include the sodium, potassium, lithium, ammonium, calcium and magnesium salts. Organic bases include primary, secondary and tertiary amines, substituted amines including naturally-occurring substituted amines, and cyclic amines, including isopropylamine, trimethyl amine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, and N-ethylpiperidine.

Acid/base addition salts tend to be more soluble in aqueous solvents than the corresponding free acid/base forms.

The present invention also provides a pharmaceutical composition comprising a therapeutically effective amount of a compound as hereinbefore defined, or a pharmaceutically acceptable salt thereof, together with at least one pharmaceutically acceptable carrier or diluent.

The term “composition” is intended to include the formulation of an active ingredient with encapsulating material as carrier, to give a capsule in which the active ingredient (with or without other carrier) is surrounded by carriers.

While the compounds as hereinbefore described, or pharmaceutically acceptable salts thereof, may be the sole active ingredient administered to the subject, the administration of other active ingredient(s) with the compound is within the scope of the invention. In one or more embodiments it is envisaged that a combination of two or more of the compounds of the invention will be administered to the subject. It is envisaged that the compound(s) could also be administered with one or more additional therapeutic agents in combination. The combination may allow for separate, sequential or simultaneous administration of the compound(s) as hereinbefore described with the other active ingredient(s). The combination may be provided in the form of a pharmaceutical composition.

The term “combination”, as used herein refers to a composition or kit of parts where the combination partners as defined above can be dosed dependently or independently or by use of different fixed combinations with distinguished amounts of the combination partners, i.e., simultaneously or at different time points. The combination partners can then, e.g., be administered simultaneously or chronologically staggered, that is at different time points and with equal or different time intervals for any part of the kit of parts. The ratio of the total amounts of the combination partners to be administered in the combination can be varied, e.g. in order to cope with the needs of a patient sub-population to be treated or the needs of the single patient which different needs can be due to age, sex, body weight, etc. of the patients.

As will be readily appreciated by those skilled in the art, the route of administration and the nature of the pharmaceutically acceptable carrier will depend on the nature of the condition and the mammal to be treated. It is believed that the choice of a particular carrier or delivery system, and route of administration could be readily determined by a person skilled in the art. In the preparation of any formulation containing the active compound care should be taken to ensure that the activity of the compound is not destroyed in the process and that the compound is able to reach its site of action without being destroyed. In some circumstances it may be necessary to protect the compound by means known in the art, such as, for example, micro encapsulation. Similarly the route of administration chosen should be such that the compound reaches its site of action.

Those skilled in the art may readily determine appropriate formulations for the compounds of the present invention using conventional approaches. Identification of preferred pH ranges and suitable excipients, for example antioxidants, is routine in the art. Buffer systems are routinely used to provide pH values of a desired range and include carboxylic acid buffers for example acetate, citrate, lactate and succinate. A variety of antioxidants are available for such formulations including phenolic compounds such as BHT or vitamin E, reducing agents such as methionine or sulphite, and metal chelators such as EDTA.

The compounds as hereinbefore described, or pharmaceutically acceptable salts thereof, may be prepared in parenteral dosage forms, including those suitable for intravenous, intrathecal, and intracerebral or epidural delivery. The pharmaceutical forms suitable for injectable use include sterile injectable solutions or dispersions, and sterile powders for the extemporaneous preparation of sterile injectable solutions. They should be stable under the conditions of manufacture and storage and may be preserved against reduction or oxidation and the contaminating action of microorganisms such as bacteria or fungi.

The solvent or dispersion medium for the injectable solution or dispersion may contain any of the conventional solvent or carrier systems for the active compound, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolarity, for example, sugars or sodium chloride. Preferably, the formulation for injection will be isotonic with blood. Prolonged absorption of the injectable compositions can be brought about by the use in the compositions of agents delaying absorption, for example, aluminium monostearate and gelatin. Pharmaceutical forms suitable for injectable use may be delivered by any appropriate route including intravenous, intramuscular, intracerebral, intrathecal, epidural injection or infusion.

Sterile injectable solutions are prepared by incorporating the compounds of the invention in the required amount in the appropriate solvent with various of the other ingredients such as those enumerated above, as required, followed by filtered sterilization. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

Other pharmaceutical forms include oral and enteral formulations of the present invention, in which the active compound may be formulated with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal or sublingual tablets, troches, capsules, elixirs, suspensions, syrups, wafers, and the like. The amount of active compound in such therapeutically useful compositions is such that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the compounds of the invention may be incorporated into sustained-release preparations and formulations, including those that allow specific delivery of the active peptide to specific regions of the gut.

Liquid formulations may also be administered enterally via a stomach or oesophageal tube. Enteral formulations may be prepared in the form of suppositories by mixing with appropriate bases, such as emulsifying bases or water-soluble bases. It is also possible, but not necessary, for the compounds of the present invention to be administered topically, intranasally, intravaginally, intraocularly and the like.

Pharmaceutically acceptable vehicles and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate the compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutically acceptable vehicle. The specification for the novel dosage unit forms of the invention are dictated by and directly dependent on (a) the unique characteristics of the active material and the particular therapeutic effect to be achieved, and (b) the limitations inherent in the art of compounding active materials for the treatment of disease in living subjects having a diseased condition in which bodily health is impaired as herein disclosed in detail.

As mentioned above the principal active ingredient may be compounded for convenient and effective administration in therapeutically effective amounts with a suitable pharmaceutically acceptable vehicle in dosage unit form. A unit dosage form can, for example, contain the principal active compound in amounts ranging from 0.25 μg to about 2000 mg. Expressed in proportions, the active compound may be present in from about 0.25 μg to about 2000 mg/mL of carrier. In the case of compositions containing supplementary active ingredients, the dosages are determined by reference to the usual dose and manner of administration of the said ingredients.

As used herein, the term “effective amount” refers to an amount of compound which, when administered according to a desired dosing regimen, provides the desired therapeutic activity. Dosing may occur once, or at intervals of minutes or hours, or continuously over any one of these periods. Suitable dosages may lie within the range of about 0.1 ng per kg of body weight to 1 g per kg of body weight per dosage. A typical dosage is in the range of 1 μg to 1 g per kg of body weight per dosage, such as is in the range of 1 mg to 1 g per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 100 mg per kg of body weight per dosage, such as up to 50 mg per body weight per dosage.

The terms “treatment” and “treating” as used herein cover any treatment of a condition or disease in an animal, preferably a mammal, more preferably a human, and includes treating any disease or disorder that is mediated by endosomal NK₁R signaling. The terms “prevention” and “preventing” as used herein cover the prevention or prophylaxis of a condition or disease in an animal, preferably a mammal, more preferably a human and includes prevention of a disease or disorder that is mediated by endosomal GPCR signaling.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The invention will now be described with reference to the following non-limiting examples:

Experimental Methods:

βarr Inhibitors

Putative G protein receptor kinase-2 (GRK2) phosphorylation sites within the intracellular

C-terminus of the mouse NKᵢR were predicted using Group Based Prediction System (http://gps.biocuckoo.org/wsresult.php?p=1). Peptides corresponding to these domains (S³⁹⁸SSFYSNM⁴⁰⁵, S³⁹⁰NSKTMTE³⁹⁷, L³⁸²TSNGSSR³⁸⁹) or control peptide (MSNSYSFS) with the N-terminal Tat membrane permeant sequence (YGRKKRRQRRR) were prepared. A phosphorylated peptide (E³³⁵MKS*T*RY*L³⁴²) was also synthesized and conjugated to Tat.

cDNAs

BRET probes NK₁R-RLuc8, KRas-Venus, Rab5a-Venus, βarr1-YFP, βarr2-YFP have been described (Kocan, M. et al., Frontiers in endocrinology 2010, 1, 12; Lan, T. H. et al., Traffic 2012, 13, 1450-1456). CytoEKAR and NucEKAR were from Addgene (plasmids 18680, 18681 respectively). Full length and truncated δ312 rat HA-NK₁R have been described (Dery, O. et al., American journal of physiology. Cell physiology 2001, 280, C1097-1106). RLuc8 fusions of these constructs were generated by removal of the stop codon by PCR and subcloning into a pcDNA3.1-RLuc8 vector.

Cell Lines, Transfection

HEK293 cells stably expressing rat NK₁R with N-terminal HA.11 epitope have been described (Roosterman, D. et al., Proceedings of the National Academy of Sciences of the United States of America 2007, 104, 11838-11843). HEK293 cells were transiently transfected using polyethylenimine (Polysciences) or FuGene (Promega). Cells were grown in Dulbecco's modified Eagle's medium (DMEM) supplemented with 5% FBS (37° C., 5% CO₂).

BRET

HEK293 cells were transfected with the following cDNAs: 1 μg NK₁R-RLuc8 or NK₁Rδ312-RLuc8+4 μg βarr1-YFP, βarr2-YFP, KRas-Venus, or Rab5a-Venus. After 48 h, cells were equilibrated in Hank's balanced salt solution (HBSS) at 37° C., and incubated with the RLuc substrate coelenterazine h (5 μM, 15 min). BRET ratios were determined using a microplate reader LUMIstar Omega (BMG LabTech) before and after challenge with SP (0.1-10 nM) or vehicle (dH₂O).

FRET Biosensors of Compartmentalized Signaling.

HEK293 cells were transfected with 55 ng/well rat NK₁R with N-terminal HA.11 epitope tag (HA-NK₁R) or CLR plus RAMP1 and 40 ng/well FRET biosensors. FRET was assessed 48 h after transfection, following serum restriction (0.5% FBS overnight). For experiments using clathrin or dynamin siRNA, cells were transfected with 55 ng/well rat HA-NK₁R, 40 ng/well FRET biosensor and 25 nM/well scrambled, clathrin or dynamin ON-TARGETplus SMARTpool siRNA (GE Dharmacon). FRET was assessed 72 h after transfection, following serum restriction (0.5% FBS overnight). Cells were equilibrated in HBSS at 37° C. and FRET was analyzed using a GE Healthcare INCell 2000 Analyzer. For GFP/RFP emission ratio analysis, cells were sequentially excited using a FITC filter (490/20) with emission measured using dsRed (605/52) and FITC (525/36) filters, and a polychroic optimized for the FITC/dsRed filter pair (Quad4). For CFP/YFP emission ratio analysis, cells were sequentially excited using a CFP filter (430/24) with emission measured using YFP (535/30) and CFP (470/24) filters, and a polychroic optimized for the CFP/YFP filter pair (Quad3). Cells were imaged every 1 min, allowing image capture of 14 wells per minute. Baseline emission ratio images were captured for 4 min. Cells were challenged with an EC₅₀ concentration of SP (1 nM), GGRP (1 nM) or vehicle, and images were captured for 20 min. Cells were then stimulated with the positive control (200 nM phorbol 12,13-dibutyrate for ERK; 200 nM phorbol 12,13-dibutyrate with phosphatase inhibitor cocktail for PKC; 10 μM forskolin, 100 μM 3-isobutyl-1-methylxanthine, 100 nM PGE₁ for cAMP) for 10 min to generate a maximal increase, and positive emission ratio images were captured for 4 min. Data were analyzed as described and expressed as emission ratios relative to baseline for each cell (F/F₀). Cells with >10% change in F/F₀ after stimulation with positive controls were selected for analysis.

Cell-Surface ELISA

HEK293 cells transiently transfected with HA-NK₁R or HA-NK₁δ312 were fixed in PFA (30 min). For analysis of total expression, cells were permeabilized using 0.5% NP-40 in TBS (30 min) after fixation. Cells were incubated in blocking buffer (1% skim milk powder, 0.1M NaHCO₃, 4 h, RT), and then anti-HA (1:5,000, Sigma overnight, 4° C.). Cells were washed and incubated with anti-mouse horseradish peroxidase-conjugated antibody (1:2,000, 2 h, RT). Cells were washed and stained using the SIGMAFAST® substrate (SigmaAldrich). Absorbance at 490 nm was measured using an EnVision plate reader (PerkinElmer Life Sciences). Values were normalized to HEK293 cells transfected with pcDNA3 or to untreated cells.

Mechanical Hyperalgesia, Nocifensive Behavior in Mice

Mice were acclimatized to the experimental apparatus and environment for 1-2 h on 2 successive days before experiments. Mechanical hyperalgesia was assessed by paw withdrawal to stimulation of the plantar surface of the hind-paw with graded von Frey filaments. On the day before the study, von Frey scores were measured in triplicate to establish a baseline for each animal. To assess edema of the paw, hindpaw thickness was measured using digital calipers before and after treatments. For intraplantar injections, mice were sedated (5% isoflurane). Capsaicin (5 μg), Complete Freund's Adjuvant (CFA, 2 mg.ml⁻¹), or vehicle (capsaicin, 20% ethanol, 10% Tween 80, 70% saline; CFA, saline) was injected subcutaneously into the plantar surface of the left hindpaw (10 μl). von Frey scores (left and right paws) and paw thickness (left paw) were measured for 30-240 min after capsaicin, and 36-40 h after injection of CFA. Results are expressed as percent pre-injected values. For assessment of nocifensive behavior, mice were sedated and formalin (4%, 10 μl) was injected subcutaneously into the plantar surface of the left hindpaw. Mice were placed in a Perspex container and nocifensive behavior (flinching, licking, biting of the injected paw) was recorded for 60 min. The total number of nocifensive events was subdivided into acute (I, 0-10 min) and tonic (II, 10-60 min) phases.

Intrathecal Injections of Peptides in Mice

Intrathecal injections (5 μl, L3/L4) were made into conscious mice. Cell penetrating NK₁R peptides (Tat-conjugated S³⁹⁸SSFYSNM⁴⁰⁵, S³⁹⁰ NSKTMTE³⁹⁷, L³⁸²TSNGSSR³⁸⁹, each 30 μM), 100 μM control peptide (Tat-conjugated MSNSYSFS), or vehicle (1% DMSO/saline) was injected intrathecally 30 min before intra-plantar injection of capsaicin or formalin, or 36 h after CFA.

Intrathecal siRNA in Mice

Cationic liposome and adjuvant anionic polymer (polyglutamate) were used to deliver siRNA. siRNA targeting mouse βarr1 (sense 5′ AGC CUU CUG CGC GGA GAA U dTdT 3′, antisense 5′ dTdT U CGG AAG ACG CGC CUC UUA 5′) plus mouse βarr2 (sense: 5′ CCU ACA GGG UCA AGG UGA A dT dT 3′, antisense: 5′ UUC ACC UUG ACC CUG UAG G dT dT 3′) or control siRNA (sense: 5′ AAG GCC AGA CGC GAA UUA U dT dT, 3′ antisense: 5′ AUA AUU CGC GUC UGG CCU U dT dT 3′) (Dharmacon) (50 ng, 0.5 μl of 100 ng.μl⁻¹ stock) was mixed with 0.5 μl of adjuvant polyglutamate (0.1 μg.μl⁻¹ stock) and 1.5 μl sterile 0.15 M NaCl. Liposome solution, cationic lipid 2-{3-[bis-(3-amino-propyl)-amino]-propylamino}-N-ditetradecylcarbamoylmethyl-acetamide (DMAPAP) and L-α-dioleoyl phosphatidylethanolamine (DOPE) (DMAPAP/DOPE, 1/1 M:M) (2.5 μl of 200 μM) was added to siRNA/adjuvant, vortexed for 1 min, and incubated (30 min, RT). The siRNA lipoplexes were administered to mice by intrathecal injection (L1 -L4, 5 μl). After behavioral testing (36 h), the spinal cord (L1-L4) was collected for analysis of βarr1 and βarr2 expression by q-PCR.

q-PCR

Mouse lumbar spinal cord (L1 -L4) was placed in RNAlater (Qiagen) and total RNA was isolated using RNeasy RNA Isolation kit (Qiagen). Total RNA (500 ng) was reverse-transcribed using Superscript™ III cDNA Synthesis Kit (Invitrogen). cDNA was amplified using Eppendorf RealPlex Real Time PCR System. Twenty microliters of amplification reaction included cDNA template, TaqMan Universal Master Mix and TaqMan Gene Expression Assays for one of the following genes (catalog no.): ARRB2 (Mm00520666_g1), ARRB1 (Mm00617540_m1), ACTB (Mm02619580_g1), Gapdh (hs00363153_m1). Samples were amplified in triplicates. The relative abundance (R) of each transcript was estimated according to the ΔCₜ method using the following formula: 2^{ΔCT}. Cₜ is the mean critical threshold at which the increase in fluorescence is the exponential. Assuming efficiency of PCR reaction was 100%, it corresponds to a 2-fold increase in amplicon amount with each cycle of PCR. With this assumption, 2^{ΔCcT} was used to calculate relative transcript abundance. These values were normalized to β-actin and GAPHD.

### Example 1: Effects of βarr siRNA on Nociception

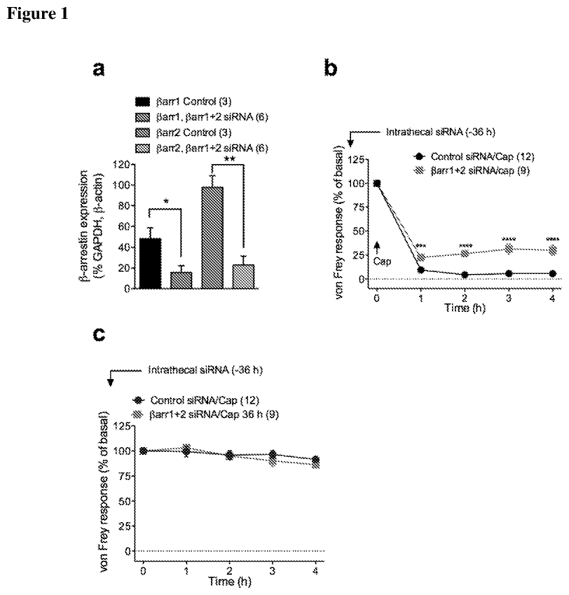

Intrathecal βarr1+2 siRNA inhibited capsaicin-evoked hyperalgesia at 36 h (FIG. 1a, b). siRNAs did not affect withdrawal responses of the uninjected paw (FIG. 1c).

### Example 2: Pharmacological Antagonism of βarr-NK₁R Interactions

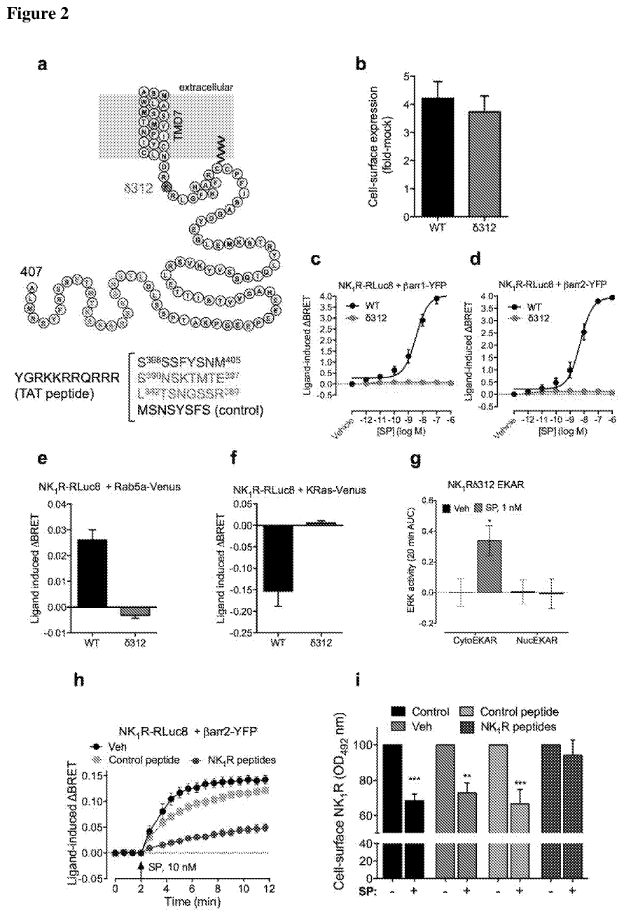

To substantiate involvement of NK₁R endocytosis in pain transmission, a pharmacological approach was devised to block NK₁R/βarr interactions. G protein receptor kinases (GRKs) phosphorylate C-terminal S/T-rich domains of GPCRs, which promotes Parr interactions. A deletion mutant NK₁Rδ312 lacks the C-terminus and corresponds to a naturally occurring NK₁R variant (Steinhoff, M. S. et al. Physiol Rev 2014, 94, 265-301). NK₁R6312 was normally expressed at the plasma membrane of HEK293 cells, but did not associate with Parrs or internalize (FIG. 2a-f). SP stimulated cytosolic but not nuclear ERK in HEK-NK₁Rδ312, consistent with endocytosis-dependent nuclear ERK signaling (FIG. 2g). Peptides corresponding to predicted GRK2 phosphorylation sites in the C-terminus of mouse NK₁R were conjugated to membrane-penetrating Tat (YGRKKRRQRRR) (FIG. 2a). These compounds inhibited association of full length NK₁R with Parrs and prevented endocytosis, compared control peptide (FIG. 2h,i).

### Example 3: Effects of NK₁R Peptides on Nociception

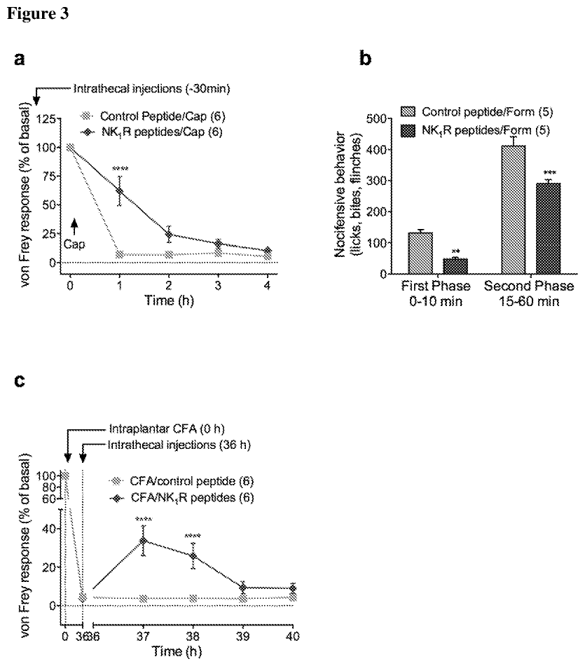

When injected intrathecally, the β-arrestin inhibitors described herein inhibited capsaicin-evoked hyperalgesia (FIG. 3a). Intrathecal β-arrestin inhibitors inhibited both phases of the nocifensive response to intraplantar formalin, and reversed the sustained mechanical hyperalgesia measured 37-40 h after intraplantar complete Freund's adjuvant (CFA) (FIG. 3b,c). Consistent with a role for NK₁R endocytosis and βarrs in SP-evoked nuclear ERK signaling (DeFea, K. A. et al. Proc Natl Acad Sci USA 2000, 97, 11086-11091), intrathecal MEK inhibitor U0126 inhibited capsaicin-evoked hyperalgesia (Ji, R. R. et al., The Journal of neuroscience : the official journal of the Society for Neuroscience 2002, 22, 478-4850).

1. A method for the treatment of a disease or disorder mediated by endosomal substance P (SP) or neurokinin 1 receptor (NK₁R) signaling comprising administering to a subject in need thereof a compound that inhibits interaction between β-arrestin and the intracellular C-terminus of the activated NK₁R.

2. A β-arrestin inhibitor of the formula:
A-D
wherein
A is a membrane permeant residue that facilitates transport of the β-arrestin inhibitor across a cellular membrane, and
D represents a fragment of one or more phosphorylation sites on the intracellular C-terminus of NK₁R, or
a pharmaceutically acceptable salt thereof.

3. A β-arrestin inhibitor according to claim 2 selected from:
A-SSSFYSNM-OH,
A-SNSKTMTE-OH,
A-LTSNGSSR-OH,
and
A-EMKS*T*RY*L-OH,
wherein
A is a membrane permeant residue that facilitates transport of the β-arrestin inhibitor across a cellular membrane; and
indicates that the amino acid residue is phosphorylated; or
a pharmaceutically acceptable salt thereof.

4. The β-arrestin inhibitor according to claim 3, wherein A is the Tat membrane permeant peptide sequence YGRKKRRQRRR.

5. The β-arrestin inhibitor according to claim 3, wherein A is palmitic acid.

6. A method for the treatment of a disease or disorder mediated by endosomal SP or NK₁R signaling comprising administering to a subject in need thereof an effective amount of a β-arrestin inhibitor according to claim 2.

7. The method according to claim 6, wherein the disease or disorder mediated by endosomal NK₁R signaling is selected from chemotherapy-induced nausea and vomiting (CINV), postoperative nausea and vomiting, affective and addictive disorders including depression and anxiety, gastrointestinal disorders including inflammatory bowel disease and irritable bowel syndrome, chronic inflammatory disorders including arthritis, respiratory disorders including COPD and asthma, urogenital disorders, sensory disorders and pain including somatic pain and visceral pain, pruritus, viral and bacterial infections and proliferative disorders (cancer).

8. The method according to claim 7, wherein the disease or disorder mediated by endosomal NK₁R signaling is somatic pain or visceral pain.

9. The method according to claim 7, wherein the disease or disorder mediated by endosomal SP or NK₁R signaling is a chronic disease or disorder.

10. (canceled)

11. (canceled)

12. The β-arrestin inhibitor according to claim 2, wherein A is the Tat membrane permeant peptide sequence YGRKKRRQRRR.

13. The β-arrestin inhibitor according to claim 2, wherein A is palmitic acid.

14. A method for the treatment of a disease or disorder mediated by endosomal SP or NK₁R signaling comprising administering to a subject in need thereof an effective amount of a β-arrestin inhibitor according to claim 3.

15. A method for the treatment of a disease or disorder mediated by endosomal SP or NK₁R signaling comprising administering to a subject in need thereof an effective amount of a β-arrestin inhibitor according to claim 4.

16. A method for the treatment of a disease or disorder mediated by endosomal SP or NK₁R signaling comprising administering to a subject in need thereof an effective amount of a β-arrestin inhibitor according to claim 5.

17. A method for the treatment of a disease or disorder mediated by endosomal SP or NK₁R signaling comprising administering to a subject in need thereof an effective amount of a β-arrestin inhibitor according to claim 12.

18. A method for the treatment of a disease or disorder mediated by endosomal SP or NK₁R signaling comprising administering to a subject in need thereof an effective amount of a β-arrestin inhibitor according to claim 13.

19. The method according to claim 1, wherein the disease or disorder mediated by endosomal NK₁R signaling is selected from chemotherapy-induced nausea and vomiting (CINV), postoperative nausea and vomiting, affective and addictive disorders including depression and anxiety, gastrointestinal disorders including inflammatory bowel disease and irritable bowel syndrome, chronic inflammatory disorders including arthritis, respiratory disorders including COPD and asthma, urogenital disorders, sensory disorders and pain including somatic pain and visceral pain, pruritus, viral and bacterial infections and proliferative disorders (cancer).

20. The method according to claim 19, wherein the disease or disorder mediated by endosomal NK₁R signaling is somatic pain or visceral pain.

21. The method according to claim 19, wherein the disease or disorder mediated by endosomal SP or NK₁R signaling is a chronic disease or disorder.


SEQUENCE LISTING (12 sequences)

SEQ ID NO: 1 - protein, 8 aa, Artificial Sequence
  Beta-arrestin inhibitor domain
SSSFYSNM

SEQ ID NO: 2 - protein, 8 aa, Artificial Sequence
  Beta-arrestin inhibitor domain
SNSKTMTE

SEQ ID NO: 3 - protein, 8 aa, Artificial Sequence
  Beta-arrestin inhibitor domain
LTSNGSSR

SEQ ID NO: 4 - protein, 8 aa, Artificial Sequence
  Beta-arrestin inhibitor domain
  MOD_RES (4)..(7): PHOSPHORYLATION, residues 4, 5, and 7
EMKSTRYL

SEQ ID NO: 5 - protein, 11 aa, Artificial Sequence
  Tat membrane premeant sequence
YGRKKRRQRRR

SEQ ID NO: 6 - protein, 8 aa, Artificial Sequence
  Control peptide
MSNSYSFS

SEQ ID NO: 7 - RNA, 21 nt, Artificial Sequence
  siRNA targeting mouse beta-arr1 sense
  misc_feature (20)..(21): n is dT
agccuucugcgcggagaaunn

SEQ ID NO: 8 - RNA, 21 nt, Artificial Sequence
  siRNA targeting mouse beta-arr1 antisense
  misc_feature (1)..(2): n is dT
nnucggaagacgcgccucuua

SEQ ID NO: 9 - RNA, 21 nt, Artificial Sequence
  siRNA targeting mouse beta-arr2 sense
  misc_feature (20)..(21): n is dT
ccuacagggucaaggugaann

SEQ ID NO: 10 - RNA, 21 nt, Artificial Sequence
  siRNA targeting mouse beta-arr2 antisense
  misc_feature (20)..(21): n is dT
uucaccuugacccuguaggnn

SEQ ID NO: 11 - RNA, 21 nt, Artificial Sequence
  control siRNA sense
  misc_feature (20)..(21): n is dT
aaggccagacgcgaauuaunn

SEQ ID NO: 12 - RNA, 21 nt, Artificial Sequence
  control siRNA antisense
  misc_feature (20)..(21): n is dT
auaauucgcgucuggccuunn